(19) United States
(12) Patent Application Publication
(10) Pub. No.: US 2026/0007815 A1
(43) Pub. Date: Jan. 8, 2026

(54) CLOSED-LOOP SYSTEM FOR OPTIMAL INSTILLATION VOLUME DETERMINATION FOR INSTILLATION WITH NEGATIVE-PRESSURE WOUND THERAPY

(71) Applicant: Solventum Intellectual Properties Company, Maplewood, MN (US)

(72) Inventors: Brett L. MOORE, San Antonio, TX (US); Elizabeth TRIMBLE, San Antonio, TX (US); Jordan TRAXLER, San Antonio, TX (US); Shannon C. INGRAM, San Antonio, TX (US); Edward LEE, San Antonio, TX (US)

(21) Appl. No.: 18/992,872

(22) PCT Filed: Jul. 12, 2023
(86) PCT No.: PCT/IB2023/057169

§ 371 Date: Jan. 9, 2025

Related U.S. Application Data
(60) Provisional application No. 63/388,867, filed on Jul. 13, 2022.

(51) Int. Cl. A61M 1/00

(52) U.S. Cl. CPC A61M 1/92; A61M 1/915; A61M 1/982; A61M 2205/18; A61M 2205/3317; A61M 2205/3327; A61M 2205/3393

(57) ABSTRACT

Systems and methods for treating a tissue site are described. The system includes an instillation source configured to provide instillation solution to the tissue site, and a negative-pressure source configured to draw fluid from the tissue site to develop a negative pressure at the tissue site. The system also includes a controller communicatively coupled to the instillation source and the negative-pressure source. The controller is configured to actuate the instillation source and actuate the negative-pressure source. The system also includes a sensor communicatively coupled to the controller and operatively coupled to the instillation source and the negative-pressure source. The sensor is configured to generate a signal indicative of an amount of fluid delivered to the tissue site and an amount of fluid recovered from the tissue site.

### CROSS-REFERENCE TO RELATED APPLICATIONS

This application is a U.S. National Stage Entry of PCT International Application No. PCT/IB2023/057169, filed on Jul. 12, 2023, which claims the benefit of priority to U.S. Provisional Application No. 63/388,867, filed on Jul. 13, 2022, which is incorporated herein by reference in its entirety.

### TECHNICAL FIELD

The invention set forth in the appended claims relates generally to system volume determinations in tissue treatment systems and more particularly, but without limitation, to determining an instillation volume of a tissue site.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for determining an optimum instillation volume in a negative-pressure environment are set forth in the appended claims. Illustrative embodiments are also provided to enable a person skilled in the art to make and use the claimed subject matter.

For example, in some embodiments a system for treating a tissue site is described. The system includes an instillation source configured to provide instillation solution to the tissue site, and a negative-pressure source configured to draw fluid from the tissue site to develop a negative pressure at the tissue site. The system also includes a controller communicatively coupled to the instillation source and the negative-pressure source. The controller is configured to actuate the instillation source and actuate the negative-pressure source. The system also includes a sensor communicatively coupled to the controller and operatively coupled to the instillation source and the negative-pressure source. The sensor is configured to generate a signal indicative of an amount of fluid delivered to the tissue site and an amount of fluid recovered from the tissue site.

More generally, a system for treating a tissue site is described. The system can include a controller communicatively coupled to an instillation source and a negative-pressure source. The controller can be configured to: actuate the instillation source to provide instillation therapy and actuate the negative-pressure source to provide negative-pressure therapy. The system can include an instillation sensor communicatively coupled to the controller and configured to generate a signal indicative of an instill volume (IV). The system can also include a recovered volume (RV) sensor communicatively coupled to the controller and configured to generate a signal indicative of a recovered volume (RV).

A method of determining saturation of a tissue site is also described herein, wherein some example embodiments provide receiving, with a controller, a signal indicative of an instill volume (IV); receiving, with the controller, a signal indicative of a recovered volume (RV); comparing, with the controller, the instill volume (IV) to the recovered volume (RV); and in response to comparing the instill volume (IV) to the recovered volume (RV), determining a saturation status of the tissue site.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is a flowchart illustrating operational steps of a process for determining an initial system volume for selection of a dressing for use with a tissue site;

FIG. 3 is a flowchart illustrating operational steps of a process performed by the system of FIG. 1 for determining an optimal fluid instillation volume of the tissue site;

FIG. 4 is a combined bar graph and line graph illustrating the determination of the optimal fluid instillation volume according to the operational steps of FIG. 3;

FIG. 5 is a flowchart illustrating operational steps of another process performed by the system of FIG. 1 for determining an optimal fluid instillation volume of the tissue site;

FIG. 6 is a combined bar graph and line graph illustrating the determination of the optimal fluid instillation volume according to the operational steps of FIG. 5;

FIG. 7 is a flowchart illustrating operational steps of another process performed by the system of FIG. 1 for determining an optimal fluid instillation volume of the tissue site;

FIG. 8 is a combined bar graph and line graph illustrating the determination of the optimal fluid instillation volume according to the operational steps of FIG. 7.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

The term “tissue site” in this context broadly refers to a wound, defect, or other treatment target located on or within tissue, including but not limited to, a surface wound, bone tissue, adipose tissue, muscle tissue, neural tissue, dermal tissue, vascular tissue, connective tissue, cartilage, tendons, or ligaments. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted. A surface wound, as used herein, is a wound on the surface of a body that is exposed to the outer surface of the body, such as injury or damage to the epidermis, dermis, and/or subcutaneous layers. Surface wounds may include ulcers or closed incisions, for example. A surface wound, as used herein, does not include wounds within an intra-abdominal cavity. A wound may include chronic, acute, traumatic, subacute, and dehisced wounds, partial-thickness burns, ulcers (such as diabetic, pressure, or venous insufficiency ulcers), flaps, and grafts, for example.

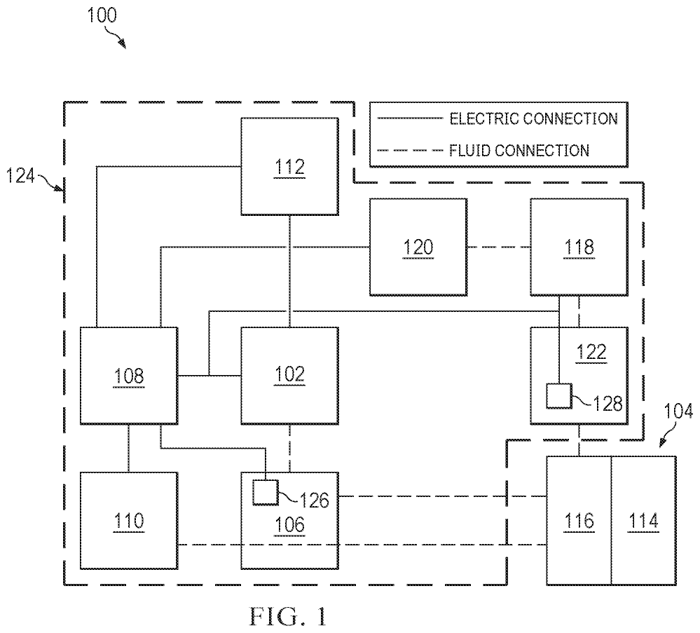

FIG. 1 is a simplified functional block diagram of an example embodiment of a therapy system 100 that can provide negative-pressure therapy with instillation of topical treatment solutions to a tissue site in accordance with this specification. The therapy system 100 may include a source or supply of negative pressure, such as a negative-pressure source 102, a dressing 104, a fluid container, such as a canister 106, and a regulator or controller, such as a controller 108, for example. Additionally, the therapy system 100 may include sensors to measure operating parameters and provide feedback signals to the controller 108 indicative of the operating parameters. As illustrated in FIG. 1, for example, the therapy system 100 may include a pressure sensor 110, an electric sensor 112, or both, coupled to the controller 108. As illustrated in the example of FIG. 1, the dressing 104 may comprise or consist essentially of a tissue interface 114, a cover 116, or both in some embodiments.

The therapy system 100 may also include a source of instillation solution. For example, a fluid source 118 may be fluidly coupled to the dressing 104, as illustrated in the example embodiment of FIG. 1. The fluid source 118 may be fluidly coupled to a positive-pressure source such as the positive-pressure source 120, a negative-pressure source such as the negative-pressure source 102, or both in some embodiments. A regulator, such as an instillation regulator 122, may also be fluidly coupled to the fluid source 118 and the dressing 104 to ensure proper dosage of instillation solution (e.g. saline or sterile water) to a tissue site. For example, the instillation regulator 122 may comprise a piston that can be pneumatically actuated by the negative-pressure source 102 to draw instillation solution from the fluid source 118 during a negative-pressure interval and to instill the solution to a dressing during a venting interval. Additionally or alternatively, the controller 108 may be coupled to the negative-pressure source 102, the positive-pressure source 120, or both, to control dosage of instillation solution to a tissue site. In some embodiments, the instillation regulator 122 may also be fluidly coupled to the negative-pressure source 102 through the dressing 104, as illustrated in the example of FIG. 1.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 102 may be combined with the fluid source 118, the controller 108, and other components into a therapy unit 124.

In general, components of the therapy system 100 may be coupled directly or indirectly. For example, the negative-pressure source 102 may be directly coupled to the canister 106, and may be indirectly coupled to the dressing 104 through the canister 106. Coupling may include fluid, mechanical, thermal, electrical, or chemical coupling (such as a chemical bond), or some combination of coupling in some contexts. For example, the negative-pressure source 102 may be electrically coupled to the controller 108, and may be fluidly coupled to one or more distribution components to provide a fluid path to a tissue site. In some embodiments, components may also be coupled by virtue of physical proximity, being integral to a single structure, or being formed from the same piece of material. For example, the tissue interface 114 and the cover 116 may be discrete layers disposed adjacent to each other, and may be joined together in some embodiments.

A distribution component is preferably detachable, and may be disposable, reusable, or recyclable. The dressing 104 and the canister 106 are illustrative of distribution components. A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 104.

A negative-pressure supply, such as the negative-pressure source 102, may be a reservoir of air at a negative pressure, or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The canister 106 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 108, may be a microprocessor or computer programmed to operate one or more components of the therapy system 100, such as the negative-pressure source 102. In some embodiments, for example, the controller 108 may be a microcontroller, which generally comprises an integrated circuit containing a processor core and a memory programmed to directly or indirectly control one or more operating parameters of the therapy system 100. Operating parameters may include the power applied to the negative-pressure source 102, the pressure generated by the negative-pressure source 102, or the pressure distributed to the tissue interface 114, for example. The controller 108 is also preferably configured to receive one or more input signals, such as a feedback signal, and programmed to modify one or more operating parameters based on the input signals.

Sensors, such as the pressure sensor 110 or the electric sensor 112, are generally known in the art as any apparatus operable to detect or measure a physical phenomenon or property, and generally provide a signal indicative of the phenomenon or property that is detected or measured. For example, the pressure sensor 110 and the electric sensor 112 may be configured to measure one or more operating parameters of the therapy system 100. In some embodiments, the pressure sensor 110 may be a transducer configured to measure pressure in a pneumatic pathway and convert the measurement to a signal indicative of the pressure measured. In some embodiments, the pressure sensor 110 may be a piezoresistive strain gauge. The electric sensor 112 may optionally measure operating parameters of the negative-pressure source 102, such as the voltage or current, in some embodiments. Preferably, the signals from the pressure sensor 110 and the electric sensor 112 are suitable as an input signal to the controller 108, but some signal conditioning may be appropriate. For example, the signal may need to be filtered or amplified before it can be processed by the controller 108. Typically, the signal is an electrical signal, but may be represented in other forms, such as an optical signal.

The tissue interface 114 can be generally adapted to partially or fully contact a tissue site. The tissue interface 114 may take many forms, and may have many sizes, shapes, or thicknesses depending on a variety of factors, such as the type of treatment being implemented or the nature and size of a tissue site. For example, the size and shape of the tissue interface 114 may be adapted to the contours of deep and irregular shaped tissue sites.

In some embodiments, the tissue interface 114 may comprise or consist essentially of a manifold. A manifold in this context may comprise or consist essentially of a means for collecting or distributing fluid across the tissue interface 114 under pressure. For example, a manifold may be adapted to receive negative pressure from a source and distribute negative pressure through multiple apertures across the tissue interface 114, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 114 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 114 may also vary according to needs of a prescribed therapy. For example, the tensile strength of foam may be increased for instillation of topical treatment solutions. The 25% compression load deflection of the tissue interface 114 may be at least 0.35 pounds per square inch, and the 65% compression load deflection may be at least 0.43 pounds per square inch. In some embodiments, the tensile strength of the tissue interface 114 may be at least 10 pounds per square inch. The tissue interface 114 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 114 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 114 may also vary according to needs of a prescribed therapy. For example, the thickness of the tissue interface may be decreased to reduce tension on peripheral tissue. The thickness of the tissue interface 114 can also affect the conformability of the tissue interface 114. In some embodiments, a thickness in a range of about 5 millimeters to 10 millimeters may be suitable.

The tissue interface 114 may be either hydrophobic or hydrophilic. In an example in which the tissue interface 114 may be hydrophilic, the tissue interface 114 may also wick fluid away from a tissue site, while continuing to distribute negative pressure to the tissue site. The wicking properties of the tissue interface 114 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 114 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 114 may further serve as a scaffold for new cell-growth, or a scaffold material may be used in conjunction with the tissue interface 114 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 116 may provide a bacterial barrier and protection from physical trauma. The cover 116 may also be constructed from a material that can reduce evaporative losses and provide a fluid seal between two components or two environments, such as between a therapeutic environment and a local external environment. The cover 116 may be, for example, an elastomeric film or membrane that can provide a seal adequate to maintain a negative pressure at a tissue site for a given negative-pressure source. The cover 116 may have a high moisture-vapor transmission rate (MVTR) in some applications. For example, the MVTR may be at least about 300 g/m² per twenty-four hours in some embodiments. In some example embodiments, the cover 116 may be a polymer drape, such as a polyurethane film, that is permeable to water vapor but impermeable to liquid. Such drapes typically have a thickness in the range of about 25 microns to about 50 microns. For permeable materials, the permeability generally should be low enough that a desired negative pressure may be maintained.

The cover 116 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; an INSPIRE 2301 material from Coveris Advanced Coatings of Wrexham, United Kingdom having, for example, an MVTR (inverted cup technique) of about 14400 g/m²/24 hours and a thickness of about 30 microns; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M Tegaderm® drape; a polyurethane (PU) drape such as one available from Avery Dennison Corporation of Glendale, California; polyether block polyamide copolymer (PEBAX), for example, from Arkema, France; INSPIRE 2327; or other appropriate material.

An attachment device may be used to attach the cover 116 to an attachment surface, such as undamaged epidermis, a gasket, or another cover. The attachment device may take many forms. For example, an attachment device may be a medically-acceptable, pressure-sensitive adhesive configured to bond the cover 116 to epidermis around a tissue site. In some embodiments, for example, some or all of the cover 116 may be coated with an adhesive, such as an acrylic adhesive, which may have a coating weight between about 25 grams per square meter (g.s.m.) and about 65 g.s.m. Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organ gel.

The fluid source 118 may also be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

The fluid mechanics of using a negative-pressure source to reduce pressure in another component or location, such as within a sealed therapeutic environment, can be mathematically complex. However, the basic principles of fluid mechanics applicable to negative-pressure therapy and instillation are generally well-known to those skilled in the art, and the process of reducing pressure may be described illustratively herein as “delivering,” “distributing,” or “generating” negative pressure, for example.

In general, exudates and other fluids flow toward lower pressure along a fluid path. Thus, the term “downstream” typically implies a position in a fluid path relatively closer to a source of negative pressure or further away from a source of positive pressure. Conversely, the term “upstream” implies a position relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications (such as by substituting a positive-pressure source for a negative-pressure source) and this descriptive convention should not be construed as a limiting convention.

Negative pressure applied across the tissue site through the tissue interface 114 in the sealed therapeutic environment can induce macro-strain and micro-strain in the tissue site. Negative pressure can also remove exudate and other fluid from a tissue site, which can be collected in canister 106.

In some embodiments, the controller 108 may receive and process data from one or more sensors, such as the pressure sensor 110 and the electric sensor 112. The controller 108 may also control the operation of one or more components of the therapy system 100 to manage the pressure delivered to the tissue interface 114. In some embodiments, controller 108 may include an input for receiving a desired target pressure and may be programmed for processing data relating to the setting and inputting of the target pressure to be applied to the tissue interface 114. In some example embodiments, the target pressure may be a fixed pressure value set by an operator as the target negative pressure desired for therapy at a tissue site and then provided as input to the controller 108. The target pressure may vary from tissue site to tissue site based on the type of tissue forming a tissue site, the type of injury or wound (if any), the medical condition of the patient, and the preference of the attending physician. After selecting a desired target pressure, the controller 108 can operate the negative-pressure source 102 in one or more control modes based on the target pressure and may receive feedback from one or more sensors to maintain the target pressure at the tissue interface 114.

In some embodiments, the controller 108 may have a continuous pressure mode, in which the negative-pressure source 102 is operated to provide a constant target negative pressure for the duration of treatment or until manually deactivated. Additionally, or alternatively, the controller may have an intermittent pressure mode. The controller 108 can operate the negative-pressure source 102 to cycle between a target pressure and atmospheric pressure. For example, the target pressure may be set at a value of 135 mmHg, for a specified period of time (e.g., 5 min), followed by a specified period of time (e.g., 2 min) of deactivation. The cycle can be repeated by activating the negative-pressure source 102, which can form a square wave pattern between the target pressure and atmospheric pressure.

In some example embodiments, the increase in negative-pressure from ambient pressure to the target pressure may not be instantaneous. For example, the negative-pressure source 102 and the dressing 104 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

The target pressure can vary with time in a dynamic pressure mode. For example, the target pressure may vary in the form of a triangular waveform, varying between a negative pressure of 50 and 135 mmHg with a rise time set at a rate of +25 mmHg/min. and a descent time set at −25 mmHg/min. In other embodiments of the therapy system 100, the triangular waveform may vary between negative pressure of 25 and 135 mmHg with a rise time set at a rate of +30 mmHg/min and a descent time set at −30 mmHg/min.

In some embodiments, the controller 108 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 108, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

In some embodiments, the controller 108 may receive and process data, such as data related to instillation solution provided to the tissue interface 114. Such data may include the type of instillation solution prescribed by a clinician, the volume of fluid or solution to be instilled to a tissue site (“fill volume”), and the amount of time prescribed for leaving solution at a tissue site (“dwell time”) before applying a negative pressure to the tissue site. The fill volume may be, for example, between 10 and 500 mL, and the dwell time may be between one second to 30 minutes. The controller 108 may also control the operation of one or more components of the therapy system 100 to instill solution. For example, the controller 108 may manage fluid distributed from the solution source 118 to the tissue interface 114. In some embodiments, fluid may be instilled to a tissue site by applying a negative pressure from the negative-pressure source 102 to reduce the pressure at the tissue site, drawing solution into the tissue interface 114. In some embodiments, solution may be instilled to a tissue site by applying a positive pressure from the positive-pressure source 120 to move solution from the solution source 118 to the tissue interface 114. Additionally, or alternatively, the solution source 118 may be elevated to a height sufficient to allow gravity to move solution into the tissue interface 114.

The controller 108 may also control the fluid dynamics of instillation by providing a continuous flow of solution or an intermittent flow of solution. Negative pressure may be applied to provide either continuous flow or intermittent flow of solution. The application of negative pressure may be implemented to provide a continuous pressure mode of operation to achieve a continuous flow rate of instillation solution through the tissue interface 114, or it may be implemented to provide a dynamic pressure mode of operation to vary the flow rate of instillation solution through the tissue interface 114. Alternatively, the application of negative pressure may be implemented to provide an intermittent mode of operation to allow instillation solution to dwell at the tissue interface 114. In an intermittent mode, a specific fill volume and dwell time may be provided depending, for example, on the type of tissue site being treated and the type of dressing being utilized. After or during instillation of solution, negative-pressure treatment may be applied. The controller 108 may be utilized to select a mode of operation and the duration of the negative pressure treatment before commencing another instillation cycle by instilling more solution.

Some therapy systems providing negative-pressure therapy and instillation therapy may encounter difficulties in determining clinically appropriate instillation volumes. Some clinicians may be unfamiliar with the technical aspects of instillation with negative-pressure wound therapy and may be unable to determine instillation volumes appropriate for individual tissue sites. Other clinicians may have their view of the tissue site obscured by an opaque dressing or wound filler. Still other clinicians may face other hindrances to the proper administration of instillation with negative-pressure wound therapy. Being unable to determine an appropriate instillation volume for a tissue site can lead to tissue site over fill. Over fill of a tissue site or dressing can refer to the instillation of a greater volume of fluid to the tissue site than the volume of the tissue site itself. Over fill can raise pressures at the tissue site and cause a dressing to fail, permitting fluid to leak from the dressing. A fluid leak caused by over fill can also cause failure of negative-pressure therapy as the dressing covering a tissue site may no longer maintain a seal during negative-pressure therapy intervals.

Failure to provide an appropriate volume of instillation solution can also lead to under fill of the tissue site. Under fill can refer to instilling a volume of fluid to the tissue site that is insufficient to properly saturate the tissue site. Under fill of instillation solution may reduce the effectiveness of instillation therapy, reducing the benefits a patient may receive from instillation therapy. Preventing over fill and under fill relies on proper estimation of a volume of a tissue site. Proper estimation of the size and volume of a tissue site can sometimes be a time consuming process. Unfortunately, the time demands placed on clinicians for patient care may exceed the number of hours in a day. Consequently, excessive time demands may lead to decreased adoption of instillation with negative-pressure wound therapy, as clinicians may avoid adoption or use of instillation with negative-pressure wound therapy. Avoiding adoption of instillation with negative-pressure wound therapy can prevent a wider population from receiving the benefits of instillation therapy with negative-pressure wound therapy.

These limitations and others may be addressed by the therapy system 100, which can provide a system and a process to determine optimum instillation volume of individual tissue sites. Determination of the optimum instillation volume of an individual tissue site allows the therapy system 100 to optimize the delivery of instillation solution, reducing the risks of over fill or under fill of the tissue site. Determination of optimum instillation volume through the use of the system 100 can also reduce clinician time demands and improve the ease of use of systems providing instillation and negative-pressure wound therapy, leading to improved patient outcomes and wider adoption of beneficial therapy.

For example, the therapy system 100 can provide instillation, solution dwell, and intervals of controlled negative pressure. In some embodiments, the controller 108 of the therapy system 100 can be equipped with instill volume determination and control logic, and a method of determining the instilled volume. The therapy system 100 further includes an apparatus for instilling a variable volume of solution, a negative pressure delivery system, and a negative pressure collection system. In some embodiments, the negative pressure collection system can be equipped with a method of determining the volume of fluid collected from the tissue site. In some embodiments, the system 100 determines an optimal instillation volume. The optimal instillation volume can be a volume that achieves saturation of the dressing 104. Saturation occurs where instillation and negative pressure cycles reach an equilibrium: the negative pressure cycle removes a same volume of liquid as the instillation cycle instilled. Equilibrium can also be expressed as the point where the dressing's retained instillate volume is maximized.

In some embodiments, the controller 108 may be communicatively coupled to the canister 106. The canister 106 may include a sensor 126 or other devices configured to determine a volume of fluid received from the tissue site. For example, the controller 108 and the sensor 126 may determine a volume of fluid received from the dressing 104 through tachometry, changes in weight, or other suitable methods. In some embodiments, the sensor 126 can be a strain gauge. As fluid enters the canister 106, the weight of the fluid added to the canister 106 can increase the force on the sensor 126. The signal generated by the sensor 126 in response to the force can be received by the controller 108 and interpreted as a weight of the fluid and a corresponding volume. In some embodiments, the controller 108 may store an intermediate value of the weight of the fluid to determine an additional amount of fluid added to the canister 106 in subsequent negative-pressure therapy cycles.

In other embodiments, the sensor 126 may be a tachometer, such as a Hall-effect sensor. For example, the sensor 126 may be disposed at a fluid inlet of the canister 106. Fluid flowing past the sensor 126 during a negative-pressure therapy cycle may cause the sensor 126 to generate a signal representative of a rotational speed. The signal may be received by the controller 108 and interpreted as a corresponding volume. In still other embodiments, the sensor 126 may be a glass level gauge or a float. The sensor 126 can be a type of hydrostatic device, such as a displacer, a bubbler, or a differential pressure transmitter. The sensor 126 can be a load cell, a magnetic level gauge, or a capacitance transmitter. In still other embodiments, the sensor 126 can be a magnetostrictive level transmitter, an ultrasonic level transmitter, a laser level transmitter, or even a radar level transmitter. The sensor 126 can also be other types of level sensors such as resistive chain, gamma ray, or microwave sensors. In an exemplary embodiment, the sensor 126 can be an optical liquid level sensor such as those produced by Strain Measurement Devices or an optical fibre water sensor such as a 44.1 WS-B/K1 or a 44.2 WS-G produced by Wolf GmbH. Other suitable sensors may be produced by SEMRAD Process Control and Monitoring, such as those produced for liquid level transmitters and indicators, and sensors produced for waste water level monitoring such as the dBi HART ultrasonic intelligent transducers produced by Pulsar Measurement can be representative of suitable types of sensors.

Preferably, the sensor 126 may be communicatively coupled to the controller 108, permitting the controller 108 to distinguish volume of fluid received each cycle of negative-pressure therapy from the volume of fluid previously contained in the canister 106. In some embodiments, the sensor 126 may be a separate flow meter fluidly coupled between the canister 106 and the dressing 104 and communicatively coupled to the controller 108 to determine a volume of fluid passing into the canister 106 each negative-pressure therapy cycle. In some embodiments, the controller 108 may be configured to adjust negative-pressure therapy in response to signals received from the sensor 126.

In some embodiments, the controller 108 can be communicatively coupled to the fluid source 118 and/or the regulator 122. The fluid source 118 and/or the regulator 122 may include a sensor 128 or other devices configured to determine a volume of fluid delivered to the dressing 104. For example, the controller 108 and the sensor 128 may determine a volume of fluid delivered to the dressing 104 through tachometry, changes in weight, or other suitable methods. In some embodiments, the sensor 128 can be a strain gauge. As fluid leaves the fluid source 118, the weight of the fluid can decrease the force on the sensor 128. The signal generated by the sensor 128 in response to the force can be received by the controller 108 and interpreted as a weight of the fluid and a corresponding volume delivered. In some embodiments, the controller 108 may store an intermediate value of the weight of the fluid to determine an additional amount of fluid removed from the fluid source 118 in subsequent instillation therapy cycles.

In other embodiments, the sensor 128 may be a tachometer, such as a Hall-effect sensor. For example, the sensor 128 may be disposed at a fluid outlet of the regulator 122. Fluid flowing past the sensor 128 during an instillation therapy cycle may cause the sensor 128 to generate a signal representative of a rotational speed. The signal may be received by the controller 108 and interpreted as a corresponding volume.

Preferably, the sensor 128 may be communicatively coupled to the controller 108, permitting the controller 108 to distinguish volume of fluid delivered each cycle of instillation therapy from the volume of fluid previously delivered through the regulator 122. In some embodiments, the sensor 128 may be a separate flow meter fluidly coupled between the regulator 122 and the dressing 104 and communicatively coupled to the controller 108 to determine a volume of fluid passing into the dressing 104 each instillation therapy cycle. In some embodiments, the controller 108 may be configured to adjust instillation therapy in response to signals received from the sensor 128.

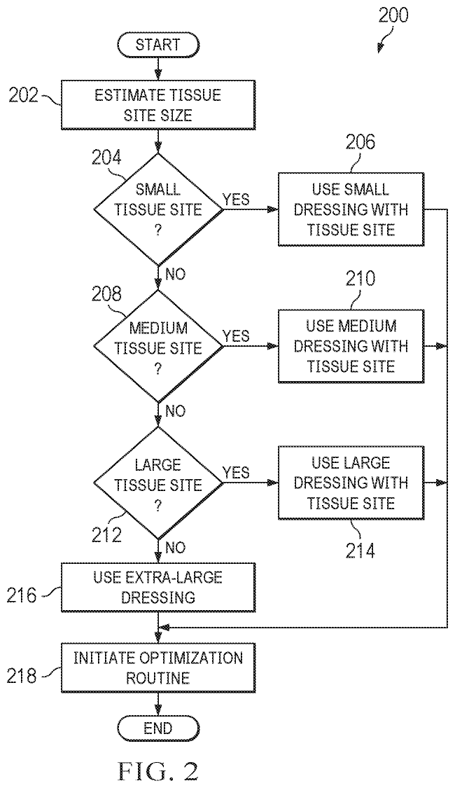

FIG. 2 is a flowchart illustrating operational steps of a process 200 for determining an initial tissue site volume for selection of a dressing for use with a tissue site. In some embodiments, a clinician may perform the steps associated with the process 200. In other embodiments, the controller 108 of the system 100 may perform the steps associated with the process 200. The process begins at block 202, where a size of the tissue site is estimated. In some embodiments, a clinician may visually estimate a size of the tissue site. For example, the clinician may visually observe the tissue site and make an initial estimate of the tissue site size. In some embodiments, the clinician may visually estimate the tissue site as small, medium, large, or extra-large. In some embodiments, the clinician may be aided by the presence of similarly named pre-packaged dressings. The clinician may visually compare the pre-packaged dressing to the tissue site to determine if the pre-packaged dressing is the appropriate size.

The process continues at block 204, where the process 200 decides if the tissue site is a small tissue site. For example, the clinician may compare a small pre-packaged dressing to the tissue site to determine if the small pre-packaged dressing may fill the tissue site. If the tissue site is a small tissue site, the process continues on the YES path to block 206, where the process 200 applies a small dressing to the tissue site. For example, the clinician may place a dressing 104 having a small volume at the tissue site. The process 200 continues to block 218, where the optimization routine is initiated. For example, the clinician may provide information to the controller 108 through the use of a user interface that a small dressing was positioned at the tissue site, and the controller 108 begins the optimization routine.

At block 204, if the tissue site is not a small tissue site, the process continues on the NO path to block 208, where the process 200 decides if the tissue site is a medium tissue site. For example, the clinician may compare a medium pre-packaged dressing to the tissue site to determine if the medium pre-packaged dressing may fill the tissue site. If the tissue site is a medium tissue site, the process continues on the YES path to block 210, where the process 200 uses a medium dressing with the tissue site. For example, the clinician may place a dressing 104 having a medium volume at the tissue site. The process 200 continues to block 218, where the optimization routine is initiated. For example, the clinician may provide information to the controller 108 through the use of a user interface that a medium dressing was positioned at the tissue site, and the controller 108 begins the optimization routine.

At block 208, if the tissue site is not a medium tissue site, the process continues on the NO path to block 212, where the process 200 decides if the tissue site is a large tissue site. For example, the clinician may compare a large pre-packaged dressing to the tissue site to determine if the large pre-packaged dressing may fill the tissue site. If the tissue site is a large tissue site, the process continues on the YES path to block 214, where the process 200 uses a large dressing with the tissue site. For example, the clinician may place a dressing 104 having a large volume at the tissue site. The process 200 continues to block 218, where the optimization routine is initiated. For example, the clinician may provide information to the controller 108 through the use of a user interface that a large dressing was positioned at the tissue site, and the controller 108 begins the optimization routine. At block 212, if the tissue site is not a large tissue site, the process continues on the NO path to block 216, where the process 200 uses an extra-large dressing with the tissue site. For example, the clinician may place a dressing 104 having an extra-large volume at the tissue site. The process 200 continues to block 218, where the optimization routine is initiated. For example, the clinician may provide information to the controller 108 through the use of a user interface that an extra-large dressing was positioned at the tissue site, and the controller 108 begins the optimization routine.

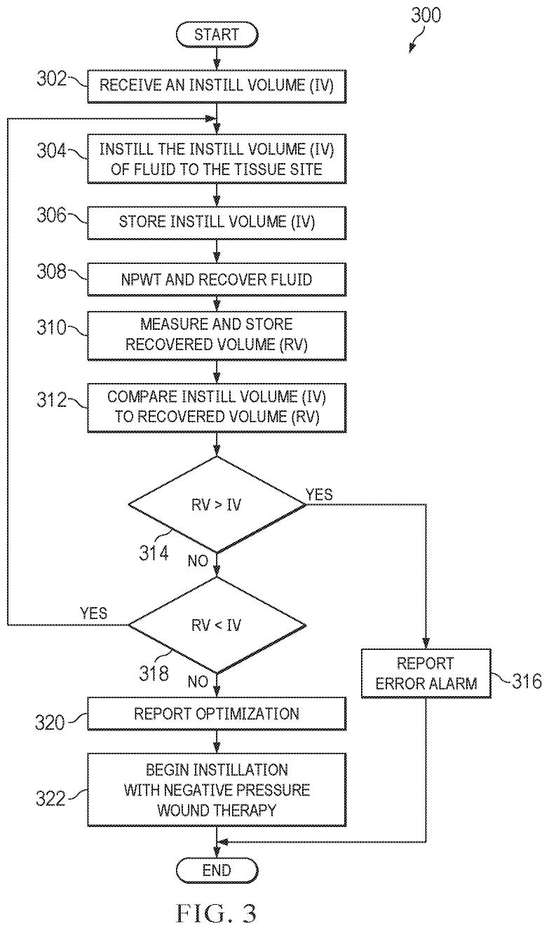

FIG. 3 is a flowchart illustrating operational steps of an optimization routine or a process 300 for determining an optimal fluid instillation volume of the tissue site performed by the system 100 of FIG. 1. In some embodiments, the process 300 may use a uniform process, a uniform instillation profile, or a uniform optimization routine. A uniform instillation profile can refer to a cyclical delivery of a same volume of fluid each instillation cycle. The process 300 begins at block 302, where the system 100 receives an instill volume (IV). In some embodiments, the controller 108 may receive an input from the process 200 of FIG. 2 to set an initial instillation volume based on the visual estimate of the tissue site size. For example, a clinician may enter that a small pre-packaged dressing was used, and the controller 108 may set the instill volume (IV) to an expected volume associated with the use of a small dressing. Similarly, if a medium, large, or extra-large pre-packaged dressing was used, the controller 108 may set the instill volume (IV) to an expected volume associated with the use of the medium, large, or extra-large pre-packaged dressing.

The process continues at block 304, where the instill volume (IV) is instilled to the tissue site. For example, the controller 108 may actuate the positive pressure source 120 and the regulator 122 to instill the instill volume (IV) to the tissue site. In some embodiments, a dwell operation may be performed following instillation of the instill volume (IV) to the tissue site. A dwell operation may be an operation permitting the instilled volume to remain in the dressing for a predetermined period of time. In other embodiments, no dwell operation is performed. The process continues at block 306, where a value associated with the instill volume (IV) is stored. For example, the sensor 128 associated with the regulator 122 and/or the fluid source 118 may provide a signal to the controller 108 indicative of a value associated with the volume of fluid instilled to the tissue site, the instill volume (IV). The controller 108 may store the value associated with the volume of fluid instilled to the tissue site or the instill volume (IV).

The process 300 continues at block 308, where fluid is drawn from the tissue site in a negative-pressure therapy cycle and a volume of fluid is recovered. For example, the controller 108 may operate the negative-pressure source 102 to draw fluid from the tissue site through the dressing 104 and into the canister 106. In some embodiments, operation of the negative-pressure source 102 may be for a cycle of negative-pressure therapy. The process continues at block 310, where the process 300 measures and stores a volume of fluid associated with the volume of fluid recovered, a recovered volume (RV). For example, the sensor 126 may generate a signal indicative of a value of the volume of fluid recovered from the tissue site, the recovered volume (RV). The controller 108 may receive and store the value associated with the signal generated by the sensor 126, the recovered volume (RV).

The process continues at block 312, where the process 300 compares the instill volume (IV) to the recovered volume (RV). For example, the controller 108 may compare the value associated with the instill volume (IV) to the value associated with the recovered volume (RV). At block 314, the process 300 decides if the recovered volume (RV) is greater than the instill volume (IV). For example, the controller 108 compares the value associated with the recovered volume (RV) to the value associated with the instill volume (IV). If the recovered volume (RV) is greater than the instill volume (IV), the process continues on the YES path to block 316, where the process 300 reports an error and the process 300 ends. For example, the controller 108 may signal an alarm, such as an auditory, tactile, or visual alarm.

At block 314, if the recovered volume (RV) is not greater than the instill volume (IV), the process 300 continues to decision block 318, where the process 300 decides if the recovered volume (RV) is less than the instill volume (IV). If the recovered volume (RV) is less than the instill volume (IV), the process 300 continues along the YES path to block 304, where the process 300 repeats. If the recovered volume (RV) is not less than the instill volume (IV), the process 300 continues on the NO path to block 320, where the process 300 reports optimization and continues to block 322. At block 322, the process 300 begins instillation with negative-pressure wound therapy. For example, the controller 108 reports, such as with an auditory, tactile, or visual indicator that the dressing 104 is at the optimum instillation volume, and the controller 108 begins instillation with negative-pressure wound therapy. In some embodiments, the controller 108 may can track a number and volume of instillations during the process 300. Once the controller 108 determines saturation has been achieved, the controller 108 can provide an optimized instillation volume for instillation with negative-pressure therapy. The optimized instillation volume can be based in part on the number and size of the instillations during the process 300 and the estimated instillation fluid left in the tissue site after each volume of fluid instilled during the process 300. The optimized instillation volume can further vary with negative pressure settings and negative pressure duration in addition to the size of the tissue site.

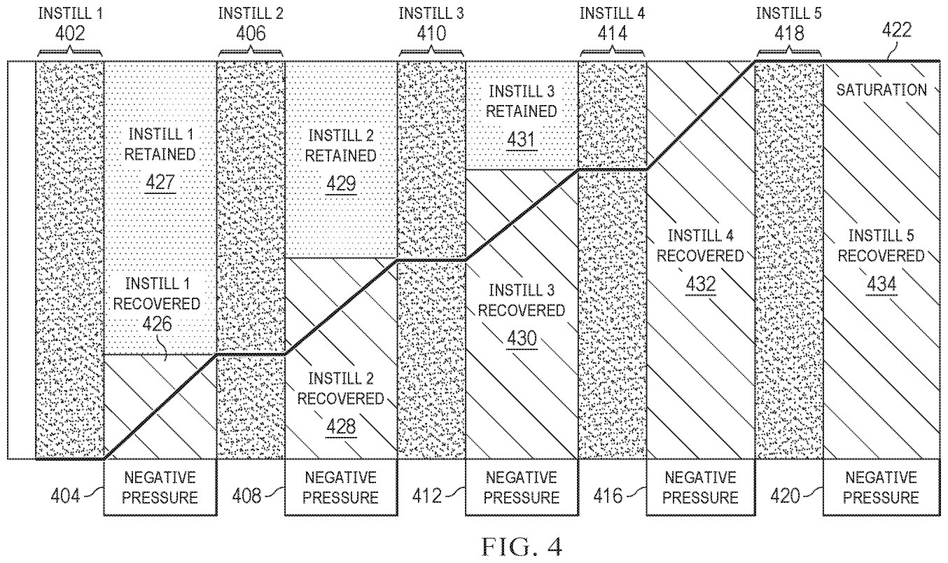

FIG. 4 is a combined bar graph and line graph illustrating fluid volume according to the operational steps of FIG. 3. In FIG. 4, the y-axis represents volume of fluid and the x- axis represents time. In each cycle through the process 300, the instill volume (IV) is the same, i.e., the volume of fluid delivered to the dressing 104 is the same. For example, in each instill cycle taking place at block 304 of the process 300, the system 100 delivers a same volume of fluid. As illustrated by bars 402, 406, 410, 414, and 418, each iteration through the process 300 delivers a same volume of fluid, i.e., the instill volume (IV) is the same. Similarly, each negative-pressure therapy cycle taking place at block 308 of the process 300 will have about a same length. For example, in each negative-pressure therapy cycle of the process 300, the system 100 will operate the negative-pressure source 102 for about a same length of time. As illustrated by bars 404, 408, 412, 416, and 420 the negative-pressure therapy cycles are about a same length. Each negative-pressure therapy cycle, the system will recover a different amount of fluid. As fluid is instilled to the tissue site, the tissue interface 114 may absorb some amount of fluid. Thus, the amount of fluid recovered during a negative-pressure therapy cycle will be the amount of fluid delivered less the amount of fluid absorbed by the tissue interface 114.

Line 422 illustrates the total aggregate volume of fluid retained by the tissue site, for example, the tissue interface 114, following each negative-pressure therapy cycle. The aggregate volume of fluid recovered following each negative-pressure therapy cycle will increase until the tissue interface 114 and the tissue site are saturated. Saturation occurs when the negative-pressure therapy cycle recovers a volume equal to the volume of fluid instilled. In the illustrated embodiment, following the negative-pressure therapy cycle illustrated by bar 404, a first volume of fluid 426 is recovered. The first volume of fluid 426 is less than the volume of fluid instilled during the instill cycle of bar 402. The remainder of the total volume of fluid instilled during instill cycle 402 is retained at the tissue site by the tissue interface 114, as illustrated by a first volume of retained fluid 427. Following the negative-pressure therapy cycle illustrated by bar 408, a second volume of fluid 428 is recovered. The second volume of fluid 428 is greater than the first volume of fluid 426 and less than the volume of fluid instilled during the instill cycle of bar 406. The remainder of the volume of fluid instilled during the instill cycle 406 is retained at the tissue site by the tissue interface 114, as illustrated by a second volume of retained 429. The second volume of retained fluid 429 is less than the first volume of retained fluid 427. Following the negative-pressure therapy cycle illustrated by bar 412, a third volume of fluid 430 is recovered. The third volume of fluid 430 is greater than the first volume of fluid 426 and the second volume of fluid 428 and less than the volume of fluid instilled during the instill cycle 410. The remainder of the volume of fluid instilled during the instill cycle 410 is retained at the tissue site by the tissue interface 114, as illustrated by a third volume of retained 431. The third volume of retained fluid 431 is less than the first volume of retained fluid 427 and the second volume of retained fluid 431. Following the negative-pressure therapy cycle illustrated by bar 416, a fourth volume of fluid 432 is recovered. The fourth volume of fluid 430 is greater than the first volume of fluid 426, the second volume of fluid 428, and the third volume of fluid 430 and equal to the volume of fluid instilled during the instill cycle of bar 414. The fourth volume of fluid 432 being equal to the amount of fluid instilled at bar 414 represents that the dressing 104 and the tissue site has reached saturation and the instillation volume is optimized. In each subsequent cycle, the volume of fluid recovered, for example, a fifth volume of fluid 434 recovered during the negative-pressure cycle 420 is substantially equal to the volume of fluid instilled prior to the negative-pressure cycle, for example, the volume of fluid instilled at bar 418.

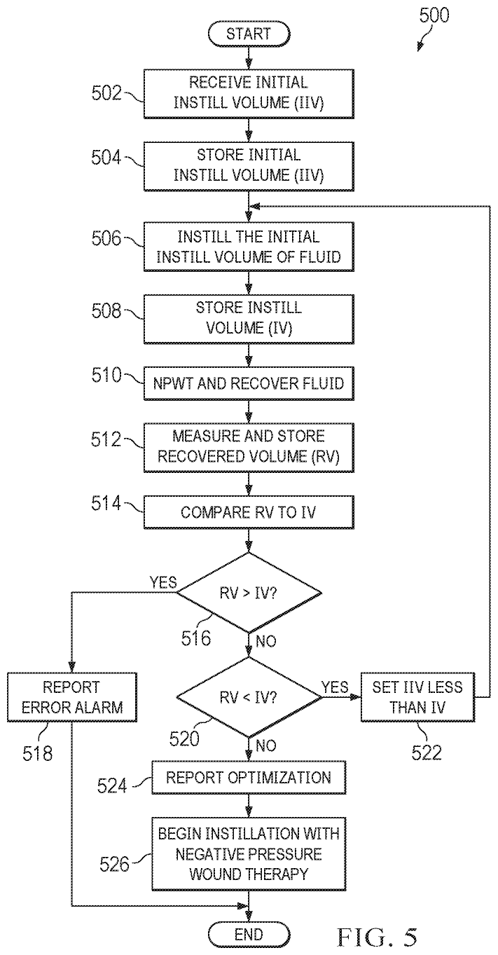

FIG. 5 is a flowchart illustrating operational steps of another optimization routine or a process 500 for determining an optimal fluid instillation volume of the tissue site performed by the system 100 of FIG. 1. In some embodiments, the process 500 may use a bolus-lead decreasing process or a bolus-lead decreasing instillation profile. The process 500 begins at block 502, where the system 100 receives an initial instill volume (IIV). In some embodiments, the process 500 receives the initial instill volume (IIV) in response to the process 200. For example, the controller 108 may receive an input from the process 200 of FIG. 2 to set an initial instillation volume (IIV) based on the visual estimate of the tissue site size. In some embodiments, a clinician may enter that a small pre-packaged dressing was used, and the controller 108 may set the initial instill volume (IIV) to an expected volume associated with the use of a small dressing. Similarly, if a medium, large, or extra-large pre-packaged dressing was used, the controller 108 may set the initial instill volume (IIV) to an expected volume associated with the use of the medium, large, or extra-large pre-packaged dressing. The process 500 continues at block 504, where the initial instillation volume (IIV) is stored. For example, the controller 108 may store a value associated with the initial instill volume (IIV).

At block 506, the initial instillation volume (IIV) is instilled to the tissue site. For example, the controller 108 may actuate the positive pressure source 120, and the regulator 122 to instill the initial instillation volume (IIV) to the dressing 104. In some embodiments, a dwell operation may be performed following instillation of the initial instill volume (IIV) to the tissue site. In other embodiments, no dwell operation is performed. The process 500 continues at block 508, where a value associated with the volume of fluid instilled to the tissue site is stored. For example, the sensor 128 may provide a signal to the controller 108 indicative of a value associated with the volume of fluid instilled to the tissue site, the instill volume (IV). The controller 108 may store the value associated with the volume of fluid instilled to the tissue site, the instill volume (IV).

The process 500 continues at block 510, where fluid is drawn from the tissue site in a negative-pressure therapy cycle and a volume of fluid is recovered, the recovered volume (RV). For example, the controller 108 may operate the negative-pressure source 102 to draw fluid from the tissue site through the dressing 104 and into the canister 106. The process 500 continues at block 512, where the process 500 measures and stores a volume of fluid associated with the recovered volume (RV). For example, the sensor 126 may generate a signal indicative of a value of the volume of fluid recovered from the tissue site, the recovered volume (RV). The controller 108 may receive and store the value associated with the signal generated by the sensor 126.

The process continues at block 514, where the process 500 compares the instill volume (IV) to the recovered volume (RV). For example, the controller 108 may compare the value associated with the instill volume (IV) to the value associated with the recovered volume (RV). At block 516, the process 500 decides if the recovered volume (RV) is greater than the instill volume (IV). For example, the controller 108 compares the value associated with the recovered volume (RV) to the value associated with the instill volume (IV). If the recovered volume (RV) is greater than the instill volume (IV), the process continues on the YES path to block 518, where the process 500 reports an error and the process 500 ends. For example, the controller 108 may signal an alarm, such as an auditory, tactile, or visual alarm.

At block 516, if the recovered volume (RV) is not greater than the instill volume (IV), the process follows the NO path to decision block 520, where the process 500 decides if the recovered volume (RV) is less than the instill volume (IV). If the recovered volume (RV) is less than the instill volume (IV), the process 500 continues along the YES path to block 522, where the process 500 iterates the volume of fluid to be instilled by setting the initial instill volume (IIV) to be less than the instill volume (IV) by a predetermined amount. For example, the controller 108 may reduce the initial instill volume (IIV) to be less than the instill volume (IV) by a predetermined amount. In some embodiments, the iteration may be selected in response to the relationship between the volume of fluid instilled and the volume of fluid recovered. For example, if the volume of fluid recovered is above a threshold percentage of the volume of fluid instilled, the system may determine that a bolus of fluid is recovered. In response, the system may increase the reduction of the subsequent instillation volume. The process continues to block 506, where the process 500 repeats.

At block 520, if the recovered volume (RV) is not less than the instill volume (IV), the process 500 continues on the NO path to block 524, where the process 500 reports optimization and continues to block 526, where the process 500 begins instillation with negative-pressure wound therapy. For example, the controller 108 reports, such as with an auditory, tactile, or visual indicator that the dressing 104 is at the optimum instillation volume, and the controller 108 begins instillation with negative-pressure wound therapy.

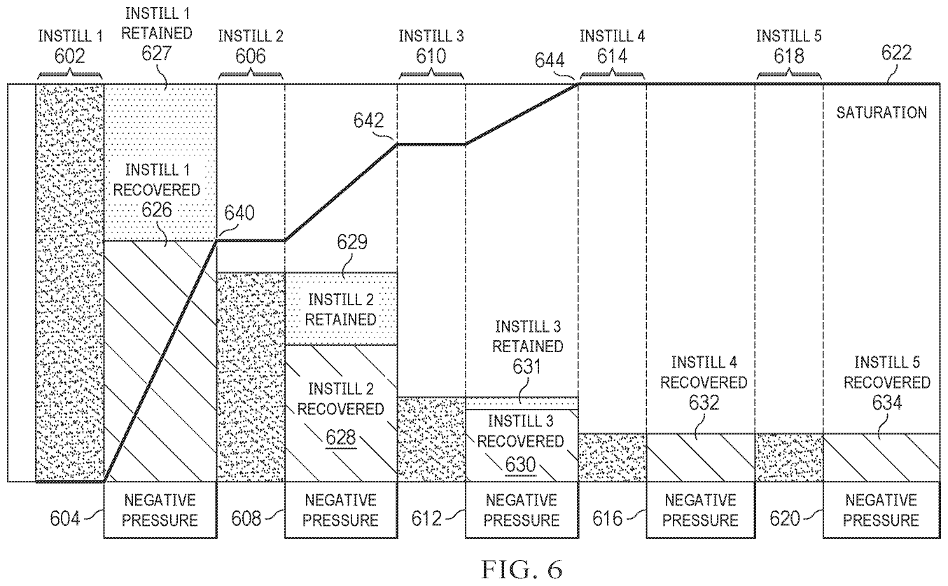

FIG. 6 is a combined bar graph and line graph illustrating the determination of the optimal fluid instillation volume according to the operational steps of FIG. 5. In FIG. 6, the y-axis represents volume of fluid and the x-axis represents time. In each cycle through the process 500, a decreasing volume is instilled to the tissue site. For example, in each subsequent instill cycle of the process 500, the system 100 delivers a smaller volume of fluid. As illustrated by bars 602, 606, 610, 614, and 616, each iteration through the process 500 delivers a smaller volume of fluid, i.e., the instill volume (IV) decreases. Each negative-pressure therapy cycle will have about a same length. For example, in each negative-pressure therapy cycle of the process 500, the system 100 will operate the negative-pressure source 102 for about a same length of time. As illustrated by bars 604, 608, 612, 616, and 620 the negative-pressure therapy cycles are about a same length. Each negative-pressure therapy cycle, the system 100 will recover a different amount of fluid. As fluid is instilled to the tissue site, the tissue interface 114 may absorb some amount of fluid. Thus, the amount of fluid recovered during a negative-pressure therapy cycle will be the amount of fluid delivered less the amount of fluid absorbed by the tissue interface 114.

Line 622 illustrates the aggregate volume of fluid retained by the tissue site, for example, the tissue interface 114, following each negative-pressure therapy cycle. The aggregate volume of fluid retained following each negative-pressure therapy cycle will increase until the tissue interface 114 and the tissue site are saturated. Saturation occurs when the negative-pressure therapy cycle recovers a volume equal to the volume of fluid instilled. In the illustrated embodiment, following the negative-pressure therapy cycle illustrated by bar 604, a first volume of fluid 626 is recovered. The first volume of fluid 626 is less than the volume of fluid instilled during the instill cycle of bar 602. The remainder of the total volume of fluid instilled during instill cycle 602 is retained at the tissue site by the tissue interface 114, as illustrated by a first volume of retained fluid 627. As illustrated in FIG. 6, the first volume of fluid 626 may be considered large; in response, the subsequent instill volume may be reduced. For example, the volume of fluid instilled during the instill cycle of bar 606 is less than the volume of fluid instilled during the instill cycle 602. Line 622 indicates at point 640 that the aggregate volume of fluid retained is substantially equal to the first volume of retained fluid 627.

Following the negative-pressure therapy cycle illustrated by bar 608, a second volume of fluid 628 is recovered. The second volume of fluid 628 is again less than the volume of fluid instilled during the instill cycle 606; however, the second volume of fluid 628 may represent a greater proportion of the volume of fluid instilled during the instill cycle 606. The remainder of the total volume of fluid instilled during instill cycle 606 is retained at the tissue site by the tissue interface 114, as illustrated by a second volume of retained fluid 629. Line 622 indicates at point 642 that the aggregate volume of fluid retained is substantially equal to the first volume of retained fluid 627 and the second volume of retained fluid 629.

Following the negative-pressure therapy cycle illustrated by bar 612, a third volume of fluid 630 is recovered. The third volume of fluid 630 is again less than the volume of fluid instilled during the instill cycle 610; however, the third volume of fluid 630 may represent a greater proportion of the volume of fluid instilled during the instill cycle 610. The remainder of the total volume of fluid instilled during instill cycle 610 is retained at the tissue site by the tissue interface 114, as illustrated by a third volume of retained fluid 631. Line 622 indicates at point 644 that the aggregate volume of fluid retained is substantially equal to the first volume of retained fluid 627, the second volume of retained fluid 629, and the third volume of retained fluid 631. Line 622 being equal to the amount of fluid instilled at bar 602 as represented at point 644 indicates that the dressing 104 and the tissue site has reached saturation and the instillation volume is optimized. Subsequent instillation cycles 614 and 618 and the respective negative-pressure cycles 616 and 620 recover volumes of fluid 632 and 634 that are substantially equal to the volume of fluid instill in the preceding instillation cycle. In response, the line 622 remains substantially horizontal, a further indication that saturation of the tissue site and the dressing 104 is reached.

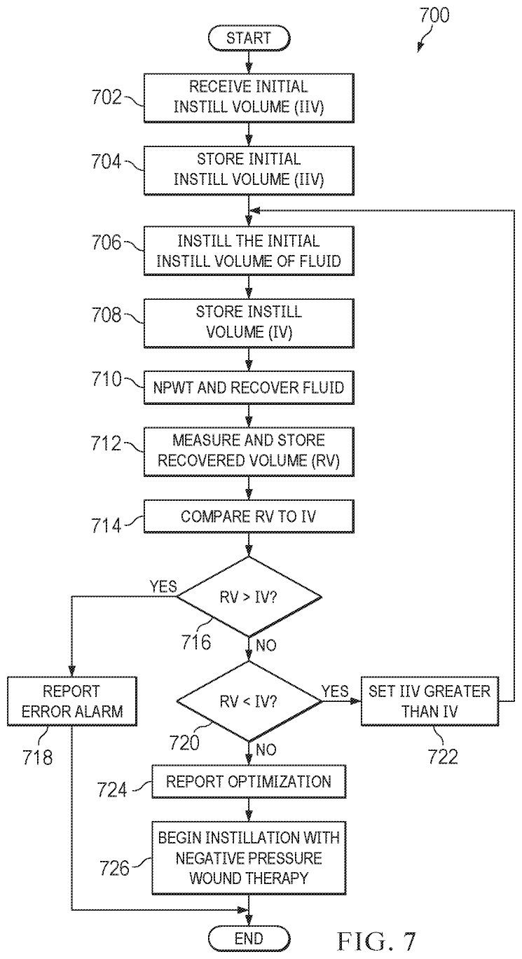

FIG. 7 is a flowchart illustrating operational steps of an optimization routine or a process 700 for determining an optimal fluid instillation volume of the tissue site performed by the system 100 of FIG. 1. In some embodiments, the process 700 may use a step-wise increasing process or a step-wise increasing instillation profile. The process 700 begins at block 702, where the system 100 receives an initial instillation volume (IIV) in response to the process 200. For example, the controller 108 may receive an input from the process 200 of FIG. 2 to set an initial instillation volume (IIV) based on the visual estimate of the tissue site size. In some embodiments, a clinician may enter that a small pre-packaged dressing was used, and the controller 108 may set the initial instill volume (IIV) to an expected volume associated with the use of a small dressing. Similarly, if a medium, large, or extra-large pre-packaged dressing was used, the controller 108 may set the initial instill volume (IIV) to an expected volume associated with the use of the medium, large, or extra-large pre-packaged dressing. The process 700 continues at block 704, where the initial instillation volume (IIV) is stored. For example, the controller 108 may store a value associated with the initial instillation volume (IIV).

At block 706, the initial instill volume (IIV) is instilled to the tissue site. For example, the controller 108 may actuate the positive pressure source 120 and the regulator 122 to instill the initial instillation volume (IIV) to the dressing 104. In some embodiments, a dwell operation may be performed following instillation of the instill volume to the tissue site. In other embodiments, no dwell operation is performed. The process 700 continues at block 708, where a value associated with the volume of fluid instilled, the instill volume (IV) is stored. For example, the regulator 122 or the fluid source 118 may provide a signal to the controller 108 indicative of a value associated with the volume of fluid instilled to the tissue site, the instilled volume (IV). The controller 108 may store the value associated with the volume of fluid instilled to the tissue site, the instilled volume (IV).

The process 700 continues at block 710, where fluid is drawn from the tissue site in a negative-pressure therapy cycle and a volume of fluid is recovered. For example, the controller 108 may operate the negative-pressure source 102 to draw fluid from the tissue site through the dressing 104 and into the canister 106. The process 700 continues at block 712, where the process 700 measures and stores a volume of fluid associated with the volume of fluid recovered, the recovered volume (RV). For example, the canister 106 may generate a signal indicative of a value of the volume of fluid recovered from the tissue site, the recovered volume (RV). The controller 108 may receive and store the value associated with the signal generated by the canister 106.

The process continues at block 714, where the process 700 compares the instill volume (IV) to the recovered volume (RV). For example, the controller 108 may compare the value associated with the instill volume (IV) to the value associated with the recovered volume (RV). At block 716, the process 700 decides if the recovered volume (RV) is greater than the instill volume (IV). For example, the controller 108 compares the value associated with the recovered volume (RV) to the value associated with the instill volume (IV). If the recovered volume (RV) is greater than the instill volume (IV), the process continues on the YES path to block 718, where the process 700 reports an error and the process 700 ends. For example, the controller 108 may signal an alarm, such as an auditory, tactile, or visual alarm.

At block 716, if the recovered volume (RV) is not greater than the instill volume (IV), the process continues to decision block 720, where the process 700 decides if the recovered volume (RV) is less than the instill volume (IV). If the recovered volume (RV) is less than the instill volume (IV), the process 700 continues along the YES path to block 722, where the process 700 iterates the volume of fluid to be instilled by setting the initial instill volume (IIV) to be greater than the instill volume (IV) by a predetermined amount. For example, the controller 108 may increase the initial instill volume (IIV) by a predetermined amount. In some embodiments, the iteration may be selected in response to the relationship between the volume of fluid instilled and the volume of fluid recovered. For example, if the volume of fluid recovered is above a threshold percentage of the volume of fluid instilled, the system may determine that a bolus of fluid is recovered. In response, the system may increase the reduction of the subsequent instillation volume. In another example, if the volume of fluid recovered is below a threshold percentage of the volume of fluid instilled, the system may determine that insufficient fluid was instilled. In response, the system may increase the subsequent instillation volume. The process continues to block 706, where the process 700 repeats.

At block 720, if the recovered volume (RV) is not less than the instill volume (IV), the process 700 continues on the NO path to block 720, where the process 700 reports optimization and continues to block 722, where the process 700 begins instillation with negative-pressure wound therapy. For example, the controller 108 reports, such as with an auditory, tactile, or visual indicator that the dressing 104 is at the optimum instillation volume, and the controller 108 begins instillation with negative-pressure wound therapy.

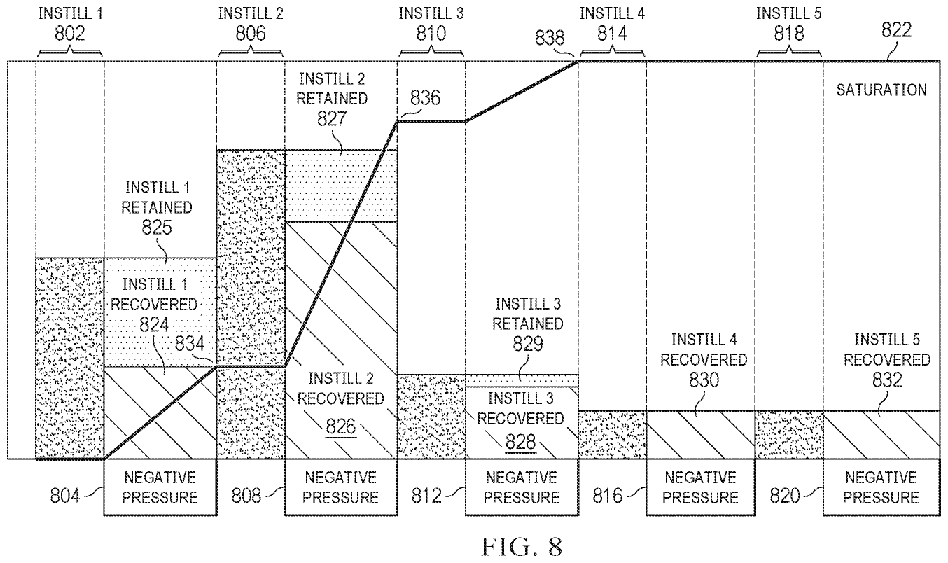

FIG. 8 is a combined bar graph and line graph illustrating the determination of the optimal fluid instillation volume according to the operational steps of FIG. 7. In FIG. 8, the y-axis represents volume of fluid and the x-axis represents time. In each cycle through the process 700, a probing volume is instilled to the tissue site. For example, in each subsequent instill cycle of the process 700, the system 100 delivers a different volume of fluid until the tissue site and the dressing 104 are saturated. Each negative-pressure therapy cycle will have about a same length. For example, in each negative-pressure therapy cycle of the process 700, the system 100 will operate the negative-pressure source 102 for about a same length of time. As illustrated by bars 804, 808, 812, 816, and 820 the negative-pressure therapy cycles are about a same length. Each negative-pressure therapy cycle, the system 100 will recover a different amount of fluid. As fluid is instilled to the tissue site, the tissue interface 114 may absorb some amount of fluid. Thus, the amount of fluid recovered during a negative-pressure therapy cycle will be the amount of fluid delivered less the amount of fluid absorbed by the tissue interface 114.

Line 822 illustrates the aggregate volume of fluid retained by the tissue site, for example, the tissue interface 114, following each negative-pressure therapy cycle. The aggregate volume of fluid retained following each negative-pressure therapy cycle will increase until the tissue interface 114 and the tissue site are saturated. Saturation occurs when the negative-pressure therapy cycle recovers a volume equal to the volume of fluid instilled. In the illustrated embodiment, following the negative-pressure therapy cycle illustrated by bar 804, a first volume of fluid 824 is recovered. The first volume of fluid 824 is less than the volume of fluid instilled during the instill cycle of bar 802. The remainder of the total volume of fluid instilled during instill cycle 802 is retained at the tissue site by the tissue interface 114, as illustrated by a first volume of retained fluid 825. As illustrated in FIG. 8, the first volume of fluid 824 may be considered small relative to the volume of fluid instilled; in response, the subsequent instill volume may be increased. For example, the volume of fluid instilled during the instill cycle of bar 806 is greater than the volume of fluid instilled during the instill cycle 802. Line 822 indicates at point 834 that the aggregate volume of fluid retained is substantially equal to the first volume of retained fluid 825.

Following the negative-pressure therapy cycle illustrated by bar 808, a second volume of fluid 826 is recovered. The second volume of fluid 826 is again less than the volume of fluid instilled during the instill cycle 806; however, the second volume of fluid 826 may represent a greater proportion of the volume of fluid instilled during the instill cycle 806. The remainder of the total volume of fluid instilled during instill cycle 806 is retained at the tissue site by the tissue interface 114, as illustrated by a second volume of retained fluid 827. Line 822 indicates at point 836 that the aggregate volume of fluid retained is substantially equal to the first volume of retained fluid 827 and the second volume of retained fluid 829. As illustrated in FIG. 8, the second volume of fluid 826 may be considered large relative to the volume of fluid instilled; in response, the subsequent instill volume may be decreased. For example, the volume of fluid instilled during the instill cycle of bar 810 is less than the volume of fluid instilled during the instill cycle 806.

Following the negative-pressure therapy cycle illustrated by bar 812, a third volume of fluid 828 is recovered. The third volume of fluid 828 is again less than the volume of fluid instilled during the instill cycle 810; however, the third volume of fluid 828 may represent a greater proportion of the volume of fluid instilled during the instill cycle 810. The remainder of the total volume of fluid instilled during instill cycle 810 is retained at the tissue site by the tissue interface 114, as illustrated by a third volume of retained fluid 829. Line 822 indicates at point 838 that the aggregate volume of fluid retained is substantially equal to the first volume of retained fluid 825, the second volume of retained fluid 827, and the third volume of retained fluid 829. Line 822 being equal to the amount of fluid instilled at bar 802 as represented at point 838 indicates that the dressing 104 and the tissue site has reached saturation and the instillation volume is optimized. Subsequent instillation cycles 814 and 818 and the respective negative-pressure cycles 816 and 820 recover volumes of fluid 830 and 832 that are substantially equal to the volume of fluid instill in the preceding instillation cycle. In response, the line 822 remains substantially horizontal, a further indication that saturation of the tissue site and the dressing 104 is reached.

The systems, apparatuses, and methods described herein may provide significant advantages. For example, the therapy system 100, which provides a process to determine an equilibrium state of the tissue site. By determining the equilibrium state of the tissue site, the therapy system 100 can optimize the delivery of instillation solution, reducing the risks of over fill or under fill of the tissue site. Optimization can also reduce clinician time demands, and improve the ease of use of systems providing instillation and negative-pressure wound therapy, leading to improved patient outcomes and wider adoption of beneficial therapy.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 104, the container 115, or both may be eliminated or separated from other components for manufacture or sale. In other example configurations, the controller 108 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for treating a tissue site, the system comprising:
an instillation source configured to provide instillation solution to the tissue site;
a negative-pressure source configured to draw fluid from the tissue site to develop a negative pressure at the tissue site;
a controller communicatively coupled to the instillation source and the negative-pressure source, the controller configured to:
actuate the instillation source; and
actuate the negative-pressure source; and
a sensor communicatively coupled to the controller and operatively coupled to the instillation source and the negative-pressure source, the sensor configured to generate a signal indicative of an amount of fluid delivered to the tissue site and an amount of fluid recovered from the tissue site.

2. The system of claim 1, wherein the controller is configured to actuate the instillation source to saturate the tissue site.

3. The system of claim 2, wherein saturation comprises drawing a same volume of fluid from the tissue site as delivered by the instillation source.

4. The system of claim 1, wherein the controller is configured to determine saturation of the tissue site.

5. The system of claim 4, wherein the controller actuates a uniform instillation profile.

6. The system of claim 5, wherein the uniform instillation profile comprises:
the controller operates the instillation source to provide an instill volume (IV);
the controller operates the negative-pressure source to recover a recovered volume (RV);
if the recovered volume (RV) is greater than the instill volume (IV), the controller generates an alarm signal;
if the recovered volume (RV) is about equal to the instill volume (IV), the controller determines that saturation is achieved and actuates the instillation source and the negative-pressure source to provide instillation therapy and negative-pressure therapy; and
if the recovered volume (RV) is less than about the instill volume (IV), the controller determines that saturation is not achieved and the controller operates the instillation source to provide the instill volume (IV).

7. The system of claim 4, wherein the controller operates the instillation source using a step-wise increasing instillation profile.

8. The system of claim 7, wherein the step-wise increasing instillation profile comprises:
the controller operates the instillation source to provide an instill volume (IV);
the controller operates the negative-pressure source to recover a recovered volume (RV);
if the recovered volume (RV) is greater than the instill volume (IV), the controller generates an alarm signal;
if the recovered volume (RV) is about equal to the instill volume (IV), the controller determines that saturation is achieved and actuates the instillation source and the negative-pressure source to provide instillation therapy and negative-pressure therapy; and
if the recovered volume (RV) is less than about the instill volume (IV), the controller determines that saturation is not achieved, and increases the instill volume (IV).

9. The system of claim 4, wherein the controller operates the instillation source using a bolus-lead instillation profile.

10. The system of claim 9, wherein the bolus-lead instillation profile comprises:
the controller operates the instillation source to provide an instill volume (IV);
the controller operates the negative-pressure source to recover a recovered volume (RV);
if the recovered volume (RV) is greater than the instill volume (IV), the controller generates an alarm signal;
if the recovered volume (RV) is about equal to the instill volume (IV), the controller determines that saturation is achieved and actuates the instillation source and the negative-pressure source to provide instillation therapy and negative-pressure therapy; and
if the recovered volume (RV) is less than about the instill volume (IV), the controller determines that saturation is not achieved, and decreases the instill volume (IV).

11. (canceled)

12. The system of claim 1, wherein:
the sensor comprises a plurality of sensors configured to determine a volume of fluid instilled and a volume of fluid recovered; and
the plurality of sensors comprise strain gauges and the controller determines a weight of the volume of fluid instilled and a weight of the volume of fluid recovered.

13. The system of claim 1, wherein;
the sensor comprises a plurality of sensors configured to determine a volume of fluid instilled and a volume of fluid recovered; and
the plurality of sensors comprise hall effect sensors and the controller determines a height of the volume of fluid instilled and a height of the volume of fluid recovered.

14. (canceled)

15. A method for determining saturation of a tissue site, the method comprising:
receiving, with a controller, a signal indicative of an instill volume (IV);
receiving, with the controller, a signal indicative of a recovered volume (RV);
comparing, with the controller, the instill volume (IV) to the recovered volume (RV); and
in response to comparing the instill volume (IV) to the recovered volume (RV), determining a saturation status of the tissue site.

16. The method of claim 15, wherein determining a saturation status of the tissue site comprises:
if the recovered volume (RV) is greater than the instill volume (IV), the controller generates an alarm signal;
if the recovered volume (RV) is about equal to the instill volume (IV), the controller determines that saturation is achieved; and
if the recovered volume (RV) is less than about the instill volume (IV), the controller determines that saturation is not achieved.

17. The method of claim 15, wherein determining a saturation status of the tissue site comprises:
if the recovered volume (RV) is greater than the instill volume (IV), the controller generates an alarm signal;
if the recovered volume (RV) is about equal to the instill volume (IV), the controller determines that saturation is achieved; and
if the recovered volume (RV) is less than about the instill volume (IV), the controller determines that saturation is not achieved, and increases the instill volume (IV).

18. The method of claim 15, wherein determining a saturation status of the tissue site comprises:
if the recovered volume (RV) is greater than the instill volume (IV), the controller generates an alarm signal;
if the recovered volume (RV) is about equal to the instill volume (IV), the controller determines that saturation is achieved; and
if the recovered volume (RV) is less than about the instill volume (IV), the controller determines that saturation is not achieved, and decreases the instill volume (IV).

19. A system for treating a tissue site, the system comprising:
a controller communicatively coupled to an instillation source and a negative-pressure source, the controller configured to:
actuate the instillation source to provide instillation therapy; and
actuate the negative-pressure source to provide negative-pressure therapy;
an instillation sensor communicatively coupled to the controller and configured to generate a signal indicative of an instill volume (IV); and
a recovered volume (RV) sensor communicatively coupled to the controller and configured to generate a signal indicative of a recovered volume (RV).

20. (canceled)

21. The system of claim 19, wherein the controller actuates a uniform instillation profile and the uniform instillation profile comprises:
the controller operates the instillation source to provide the instill volume (IV);
the controller operates the negative-pressure source to recover the recovered volume (RV);
if the recovered volume (RV) is greater than the instill volume (IV), the controller generates an alarm signal;
if the recovered volume (RV) is about equal to the instill volume (IV), the controller determines that saturation is achieved and actuates the instillation source and the negative-pressure source to provide instillation therapy and negative-pressure therapy; and
if the recovered volume (RV) is less than about the instill volume (IV), the controller determines that saturation is not achieved and the controller operates the instillation source to provide the instill volume (IV).

22. (canceled)

23. The system of claim 19, wherein the controller actuates a step-wise increasing instillation profile and the step-wise increasing instillation profile comprises:
the controller operates the instillation source to provide the instill volume (IV);
the controller operates the negative-pressure source to recover the recovered volume (RV);
if the recovered volume (RV) is greater than the instill volume (IV), the controller generates an alarm signal;
if the recovered volume (RV) is about equal to the instill volume (IV), the controller determines that saturation is achieved and actuates the instillation source and the negative-pressure source to provide instillation therapy and negative-pressure therapy; and
if the recovered volume (RV) is less than about the instill volume (IV), the controller determines that saturation is not achieved, and increases the instill volume (IV).

24. (canceled)

25. The system of claim 19, wherein the controller actuates a bolus-lead instillation profile and the bolus-lead instillation profile comprises:
the controller operates the instillation source to provide the instill volume (IV);
the controller operates the negative-pressure source to recover the recovered volume (RV);
if the recovered volume (RV) is greater than the instill volume (IV), the controller generates an alarm signal;
if the recovered volume (RV) is about equal to the instill volume (IV), the controller determines that saturation is achieved and actuates the instillation source and the negative-pressure source to provide instillation therapy and negative-pressure therapy; and
if the recovered volume (RV) is less than about the instill volume (IV), the controller determines that saturation is not achieved, and decreases the instill volume (IV).

26. (canceled)
